(19) United States
(12) Patent Application Publication
(10) Pub. No.: US 2018/0305370 A1
(43) Pub. Date: Oct. 25, 2018

(54) ETHYLENE GLYCOL ETHER OF BUPRENORPHINE

(71) Applicant: ORPHOMED, INC., Mill Valley, CA (US)

(72) Inventor: Nikhilesh Nihala SINGH, Mill Valley, CA (US)

(21) Appl. No.: 15/767,320

(22) PCT Filed: Oct. 25, 2016
(86) PCT No.: PCT/US2016/058594

§ 371 Date: Apr. 10, 2018

Related U.S. Application Data
(60) Provisional application No. 62/246,211, filed on Oct. 26, 2015.

(51) Int. Cl. C07D 489/12; A61P 25/22; A61P 25/24; A61P 29/02

(52) U.S. Cl. CPC C07D 489/12; A61P 29/02; A61P 25/24; A61P 25/22

(57) ABSTRACT

A novel ethylene glycol ether of buprenorphine for oral administration and its the treatment of chronic pain.

### CROSS-REFERENCES TO RELATED APPLICATIONS

This application claims the benefit of priority under 35 U.S.C. § 119(e) of U.S. Provisional Application No. 62/246,211 filed Oct. 26, 2015, the disclosure of which is incorporated herein by reference.

### STATEMENT AS TO RIGHTS TO INVENTIONS MADE UNDER FEDERALLY SPONSORED RESEARCH AND DEVELOPMENT

Not Applicable

### REFERENCE TO A “SEQUENCE LISTING,” A TABLE, OR A COMPUTER PROGRAM LISTING APPENDIX SUBMITTED ON A COMPACT DISK

Not Applicable

### BACKGROUND OF THE INVENTION

Chronic pain is the primary reason for health care access in the United States, with upwards of 100 million patients. It is inadequately managed in over 60 percent of such patients with consequently reduced quality of life, diminished functional ability and loss of productivity. There are several categories of medications used to treat chronic pain. Chronic opioid therapy (COT) is usually reserved for those who have intractable chronic pain that is not adequately managed with more conservative or interventional methods. With chronic pain, the goal of treatment is to reduce pain and improve function, so the patient can resume day-to-day activities.

There are several issues, however, with available opioid therapy including safety and tolerability, potential for abuse, misuse and diversion and route of administration and dosage form limitations. The ideal treatment for patients with chronic pain should provide continuous relief of pain with minimal or no harmful side effects. It should be easy and convenient to administer. Orally available (swallowed) medication available in several dosage strengths for individual titration to the most appropriate dose would meet these easy and convenient dosing criteria.

Oral dosing is critical for the treatment of chronic pain because it is convenient and facilitates dose titration when patient is not directly under medical supervision. Dose titration is essential because of the heterogeneous nature of chronic pain. Different doses are often required to treat different types of pain. Dose titration often becomes necessary not only at the beginning of treatment to assure that the patient is receiving the most effective and safest dose, but also during the course of treatment because of variations in pain intensity that often occur over time.

The opioid buprenorphine (Formula I), a partial μ agonist and a full κ antagonist, could be that ideal analgesic agent for the treatment of chronic pain. It has the structure:

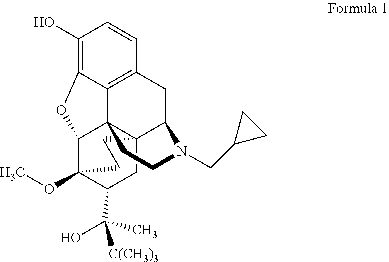

Buprenorphine is a potent analgesic and typically is about thirty times more potent an analgesic than morphine. The drug has a low potential for abuse, and due to its partial μ agonist activity, has a ceiling effect on respiratory depression with increasing dose. Additionally, buprenorphine has dual therapeutic activity, i.e., treatment of analgesia at a dose typically about a tenth of the dose required for opioid dependence.

Unfortunately, buprenorphine cannot be administered orally like most of the other opioids because it is deactivated in the gastrointestinal tract by phase 2 metabolism enzymes to form water soluble ester metabolites, O-glucuronide and sulphate esters with the phenolic hydroxyl group of buprenorphine. Consequently, absolute bioavailability of buprenorphine after an oral dose is inconsistent and is less than 10%. Therefore, it is not a surprise that there is not an FDA approved oral formulation of buprenorphine for treatment of either of opioid dependence or pain (Table 1).

TABLE 1
Buprenorphine dosage forms approved by the US FDA

| | | Approval | |
|---|---|---|---|
| Product | Formulation | Date | Indication |
| Buprenex ™* | Injection | 1981 | Moderate to severe |
| | | | pain |
| Subutex ™* | Sublingual | 2002 | Opioid dependence |
| Suboxone ™* | Sublingual with | 2002 | Opioid dependence |
| | naloxone | | |
| Butrans ™ | Transdermal patch | 2010 | Moderate to severe |
| | | | pain |
| Bunavail ™ | Buccal patch with | 2014 | Opioid dependence |
| | naloxone | | |

*Generics available

Receptor pharmacology supports the unique therapeutic properties of buprenorphine (Table 2):

TABLE 2
Opioid receptor pharmacology of buprenorphine and its therapeutic uses

| Opioid | | | |
|---|---|---|---|
| receptor | Buprenorphine (Ki) | Activity | Effect |
| μ | 0.08 | Partial agonist with | Anti-nociception |
| | | slow receptor | Ceiling effect on |
| | | dissociation | respiratory |
| | | | depression |
| δ | 0.42 | Antagonist | No application |
| | | | known |
| κ | 0.11 | Antagonist | Peripheral anti- |
| | | | hyperalgesia |
| ORL-1 | 285 | Agonist | Reduces rewarding |
| | | | behavior |

### BRIEF SUMMARY OF THE INVENTION

We have now synthesized the ethylene glycol ether (“EGE”) of buprenorphine. Its structure is:

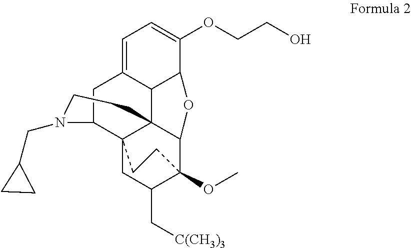

The compound's name is: 2-[(2S)-2-[(5R,6R,7R,14S)-9α-cyclopropylmethyl-4,5-epoxy-6,14-ethano-3-hydroxy-6-methoxymorphinan-7-yl]-3,3-dimethylbutan-2-ol]-ethanol. Its molecular weight is 512.

EGE buprenorphine is absorbed from the gastrointestinal tract after oral administration and, unlike buprenorphine, the compound does not undergo significant first-pass metabolism. The opioid receptor pharmacology of the compound of the invention is similar to that of buprenorphine but unlike the latter, its absorption into the systemic circulation and rapid metabolism permits its delivery by oral administration.

Owing to its receptor pharmacology, the compound of the invention may be employed orally for the pain therapy for which buprenorphine would be suited, but for its diminished bioavailability when so delivered. As an oral agent available in different dosage strengths, it can be easily titrated to the lowest effective and safe dose and be useful in the treatment of pain under various circumstances, i.e., when non-opioid analgesics are no longer effective or cannot be utilized or when increasing doses become necessary for pain control. It can also be formulated with naloxone as an abuse deterrent.

Remarkably, the compound of the invention is significantly more stable than the corresponding diethylene glycol ether, which we also synthesized for comparative purposes.

In one embodiment, the invention provides a novel ethylene glycol ether of buprenorphine for oral administration in the treatment of chronic pain.

In another aspect, provided herein, is a method of treating chronic pain in a patient. The method includes administering to the patient a therapeutically effective amount of a pharmaceutical composition comprising a pharmaceutically acceptable carrier or excipient and EGE buprenorphine having Formula 2 above or a solvate or salt thereof.

In another aspect, provided herein, is a method of treating chronic anxiety and depression in a patient. The method includes administering to the patient a therapeutically effective amount of a pharmaceutical composition comprising a pharmaceutically acceptable carrier or excipient and EGE buprenorphine having Formula 2 above or a solvate or salt thereof.

The EGE buprenorphine compounds may be combined for administration with a pharmaceutically acceptable excipient or carrier.

The EGE buprenorphine free base pictured above may be employed as such or in the form of a pharmaceutically acceptable salt or solvate.

The pharmaceutical composition may be formulated as an oral tablet, capsule or film, or extended release oral tablet, capsule or film.

Other objects, features, and advantages of the present invention will be apparent to one of skill in the art from the following detailed description and figures.

### BRIEF DESCRIPTION OF THE DRAWINGS

FIG. 1 depicts the microsomal metabolism of the compound of the invention.

FIG. 2 depicts the opioid receptor binding profile of the compound of the invention—human μ opioid receptor binding profile.

FIG. 3 depicts the opioid receptor binding profile of the compound of the invention—human μ opioid receptor binding profile.

FIG. 4 depicts the opioid receptor functionality assay of the compound of the invention—human μ opioid receptor agonist function assay.

FIG. 5 depicts the opioid receptor functionality assay of the compound of the invention—Human μ opioid receptor antagonist function assay.

FIG. 6 depicts the profile of the compound of the invention after IV and oral dose.

FIG. 7 illustrates the stability of the compound of the invention over time.

FIG. 8 illustrates the relative instability of diethylene glycol ether of buprenorphine over time.

FIG. 9 illustrates the analgesic effect of EGE buprenorphine hydrochloride in a formalin induced rat paw pain model.

### DETAILED DESCRIPTION OF THE INVENTION

### Definitions

When describing the compounds, compositions, methods and processes of this invention, the following terms have the following meanings, unless otherwise indicated.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The term “acute pain” refers to pain persisting for less than 3 to 6 months.

The term “administering,” “administration” and derivatives thereof refers to the methods that may be used to enable delivery of agents or compositions to the desired site of biological action.

The term “chronic pain” refers to pain persisting for an extended period of time, for example, greater than three to 6 months, although the characteristic signs of pain can occur earlier or later than this period. Chronic pain may be mild, excruciating, episodic, or continuous.

The term “composition” as used herein is intended to encompass a product comprising the specified ingredients in the specified amounts, as well as any product, which results, directly or indirectly, from combination of the specified ingredients in the specified amounts.

The term “pharmaceutically acceptable” carrier, diluent, or excipient is a carrier, diluent, or excipient compatible with the other ingredients of the formulation and not deleterious to the recipient thereof.

The term “subject,” “individual” or “patient” refers to an animal such as a mammal, including, but not limited to, primates (e.g., humans), cows, sheep, goats, horses, dogs, cats, rabbits, rats, mice and the like.

The term “therapeutically effective amount” refers to that amount of the therapeutic agent, which yields an appreciable and beneficial effect on the treated indication.

The term “treating,” “treatment” and derivatives thereof to refers to the treating or treatment of a disease or medical condition (such as pain) in a patient, such as a mammal (particularly a human or an animal) which includes: ameliorating the disease or medical condition, i.e., eliminating or causing regression of the disease or medical condition in a patient; suppressing the disease or medical condition, i.e., slowing or arresting the development of the disease or medical condition in a patient; or alleviating the symptoms of the disease or medical condition in a patient.

The pharmaceutical compositions disclosed herein may comprise a pharmaceutically acceptable carrier. In certain aspects, pharmaceutically acceptable carriers are determined in part by the particular composition being administered, as well as by the particular method used to administer the composition. Accordingly, there is a wide variety of suitable formulations of pharmaceutical compositions of the present invention (see, e.g., Remington's Pharmaceutical Sciences, 18th Ed., Mack Publishing Co., Easton, Pa. (1990)).

As will be appreciated, a pharmaceutically acceptable salt may be used instead of or in addition to EGE buprenorphine in any or all of the compositions and methods of treating discussed herein. Thus, in specific embodiments, a pharmaceutically acceptable salt of EGE buprenorphine (i.e., any pharmaceutically acceptable salt) is used in the methods of the invention. These salts can be prepared, for example, in situ during the final isolation and purification of the compound or by reacting separately the purified compound in its free base form with a suitable organic or inorganic acid and isolating the salt thus formed. In some embodiments, the pharmaceutically acceptable salt of EGE is prepared using acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, or p-toluenesulfonic acid. For further description of pharmaceutically acceptable salts that can be used in the methods described herein see, for example, S. M. Berge et al., “Pharmaceutical Salts,” 1977, J. Pharm. Sci. 66:1-19, which is incorporated herein by reference in its entirety.

The compound of the invention can exist in unsolvated as well as solvated forms with pharmaceutically acceptable solvents such as water, ethanol, and the like. In general, the solvated forms are considered equivalent to the unsolvated forms for the purposes of the present invention. In a specific embodiment, the solvated form of EGE is a hydrate.

In general, salt formation may improve shelf life of the resultant therapeutic agent. Appropriate salt synthesis can afford products that are crystalline, less prone to oxidation and easy to handle. Various salts can be prepared that would afford stable and crystalline compounds. A few examples are hydrochloric, sulfuric, p-toluenesulfonic, methanesulfonic, malonic, fumaric, and ascorbic acid salts.

In certain specific embodiments, such a pharmaceutical composition is formulated as oral tablet or capsule, extended release oral tablet or capsule (hard gelatin capsule, soft gelatin capsule), sublingual tablet or film, or extended release sublingual tablet or film. Illustrative pharmaceutically acceptable carriers and formulations are described in more detail below.

### Dosing and Routes of Administration

The pharmaceutical compositions of the invention are administered in a manner compatible with the dosage formulation, and in such amount as will be therapeutically effective to ameliorate pain, e.g., acute or chronic pain or opioid dependence, in the latter case commonly in conjunction with an inverse μ a opioid receptor agonist, e.g., naloxone. The quantity to be administered depends on a variety of factors including, e.g., the age, body weight, physical activity, and diet of the individual, and the stage or severity of the treated condition. In certain embodiments, the size of the dose may also be determined by the existence, nature, and extent of any adverse side effects that accompany the administration of a therapeutic agent in a particular individual.

In general, dosing of the oral form of EGE buprenorphine will be informed by current practice with buprenorphine itself and by that with orally active opioids such as morphine, factored by the different bioavailability and efficacy of EGE buprenorphine. In animal studies versus morphine (Example 7 below) EGE buprenorphine hydrochloride exhibited about 40% less bioavailability and about 2.5% less activity than morphine.

For pain in adult subjects not tolerant to opioids (e.g., subjects not opioid dependent) preferred dose may range from about 0.3 mg to about 16 mg/day, more preferably about 1 mg to about 8 mg/day, most preferably given in equally divided doses every 6 hours. Pediatric doses for pain will preferably be toward the lower end of these ranges.

For chronic anxiety and depression in adult subjects not tolerant to opioids (e.g., subjects not opioid dependent) preferred dose may range from about 0.3 mg to about 48 mg/day, more preferably about 1 mg to about 16 mg/day, most preferably given in equally divided doses every 6 hours. Pediatric doses for pain will preferably be toward the lower end of these ranges.

To deter abuse, the EGE buprenorphine may be given in combination with a therapeutically effective amount of an inverse μ agonist, e.g., naloxone or naltrexone, in the ratio of about 1:1, 2:1, 3:1. or 4:1, inverse μ agonist to EGE buprenorphine.

### Pharmaceutical Compositions

Compositions according to the invention can be administered to a subject orally in the conventional form of preparations, such as capsules, microcapsules, tablets, granules, powder, troches, pills, suppositories, oral suspensions, syrups, oral gels, sprays, solutions and emulsions. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol).

Liquid dosage forms can be prepared by dissolving or dispersing EGE compound and optionally one or more pharmaceutically acceptable adjuvants in a carrier such as, for example, aqueous saline (e.g., 0.9% w/v sodium chloride), aqueous dextrose, glycerol, ethanol, and the like, to form a solution or suspension, e.g., for oral administration. In some embodiments, the liquid dosage form is sterile.

The therapeutically effective dose can also be provided in a lyophilized form. Such dosage forms may include a buffer, e.g., bicarbonate, for reconstitution prior to administration, or the buffer may be included in the lyophilized dosage form for reconstitution with, e.g., water.

Methods for preparing such dosage forms are known to those skilled in the art (see, e.g., Remington's Pharmaceutical Sciences, 18th Ed., Mack Publishing Co., Easton, Pa. (1990)). The dosage forms typically include a conventional pharmaceutical carrier or excipient and may additionally include other medicinal agents, carriers, adjuvants, diluents, tissue permeation enhancers, solubilizers, and the like. Appropriate excipients can be tailored to the particular dosage form by methods well known in the art (see, e.g., Remington's Pharmaceutical Sciences, supra).

In addition, the compound of the invention may be formulated, alone or together, in suitable dosage unit formulations containing conventional nontoxic pharmaceutically acceptable carriers, adjuvants and vehicles appropriate for each rouse of administration.

### Example 1

### Synthesis

The hydrochloride salt of EGE buprenorphine was synthesized in 3 steps:

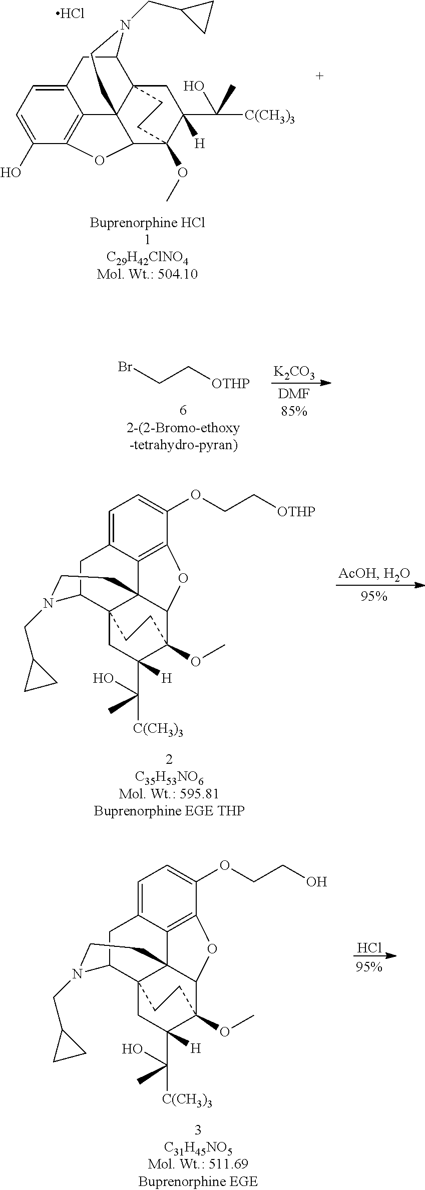

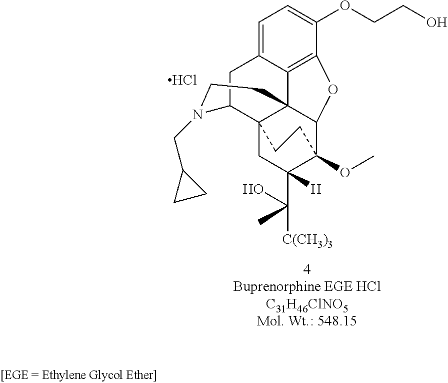 [SMILES: Cl ; CO[C@@]12CC[C@]3(C[C@H]1[C@@](C)(O)C(C)(C)C)C1Cc4ccc(OCCO)c5c4[C@]3(CCN1CC1CC1)C2O5]

Step 1—synthesis of intermediate 2: A 250-mL, three-neck round bottom flask equipped with a magnetic stirrer, addition funnel, and nitrogen inlet was charged with buprenorphine HCl (5.0 g, 10.68 mmol, 1 equiv), anhydrous DMSO (30 mL) and powdered potassium carbonate (2.94 g, 21.37 mmol, 2 equiv). The resulting mixture was heated to 55° C. and 2-(2-bromoethoxy)tetrahydro-2H-pyran (Intermediate 6) diluted with anhydrous DMSO (20 mL) was added dropwise via addition funnel over a period of 1 hour. This mixture was heated at 55° C. overnight. TLC indicated the reaction is complete. The reaction was cooled to room temperature, diluted with dichloromethane (10 vol) and washed with water (15 vol). The organic layer was separated, washed with brine, dried over magnesium sulfate and concentrated. The crude product was column chromatographed (0-5% MeOH/DCM) to yield the product 2 as a foamy solid (5.4 g, 85%). ¹H NMR is consistent.

Step 2—synthesis of intermediate 3: A 250-mL, three-neck round bottom flask equipped with a magnetic stirrer, addition funnel, and nitrogen inlet was charged with intermediate 2 (10 g, 16.78 mmol, 1.0 equiv). 100 mL of methanol/acetic acid/water (7:3:1) was added and the mixture and heated at 55° C. overnight. The reaction was complete by TLC analysis (5% MeOH/DCM) and cooled to room temperature. The reaction mixture was diluted with 100 mL of water and aqueous sodium bicarbonate solution was added dropwise to neutralize the remaining acetic acid. The resulting mixture was extracted with dichloromethane and the organic layer separated, washed with brine, dried over magnesium sulfate and concentrated to yield a viscous oil. The crude product was column chromatographed (0-5% MeOH/DCM) to yield the product 3 as a foamy solid (8.2 g, 95%). ¹H NMR is consistent.

Step 3—synthesis of ethylene glycol ether derivative of buprenorphine: A 250-mL, three-neck round bottom flask equipped with a magnetic stirrer, addition funnel, and nitrogen inlet was charged with compound 3 (8.2 g, 16 mmol) was dissolved in ethyl acetate (41 mL, 5 vol) and HCl in 1,4-dioxane (1.2 equivalent) was added dropwise to initiate precipitation. The mixture was stirred at room temperature for 30 min and the solid collected via vacuum filtration, washed with ethyl acetate, and dried under reduced pressure to afford product 4 as off white solid (8.4 g, 95%).

The diethylene glycol ether conjugate of buprenorphine was similarly synthesized, using 2-[2-(2-bromoethoxy)ethoxy)tetrahydro-2H-pyran in lieu of reactant 6 above.

### Example 2

### In Vitro Assay: Metabolic Stability of EGE Buprenorphine

Incubations of EGE Buprenorphine hydrochloride (e.g., 1 μM) with human liver microsomes (e.g., 1 mg protein/mL) were carried out using a Tecan Liquid Handling System (Tecan), or equivalent, at 37±1° C. in 0.2-mL incubation mixtures (final volume) containing potassium phosphate buffer (50 mM, pH 7.4), MgCl2 (3 mM) and EDTA (1 mM, pH 7.4) with and without a cofactor, NADPH-generating system, at the final concentrations indicated in a 96-well plate format. The NADPH-generating system consisted of NADP (1 mM, pH 7.4), glucose-6-phosphate (5 mM, pH 7.4) and glucose-6-phosphate dehydrogenase (1 Unit/mL). EGE Buprenorphine was dissolved in aqueous methanolic solution (methanol 0.5% v/v, or less). Reactions were started typically by addition of the cofactor, and stopped at four designated time points (e.g., up to 120 min) by the addition of an equal volume of stop reagent (e.g., acetonitrile, 0.2 mL containing an internal standard). Zero-time incubations served as 100% value to determine percent loss of substrate. Incubations were carried out in triplicate with an exception for zero-time samples (which were incubated in quadruplicate). Zero-cofactor (no NADPH) incubations were performed at zero-time and the longest time point. The samples were subjected to centrifugation (e.g., 920×g for 10 min at 10° C.) and the supernatant fractions analyzed by LC-MS/MS. Additional incubations were carried out with microsomes in which were replaced with a marker substrate (e.g., dextromethorphan to monitor substrate loss) as positive controls to determine if the test system is metabolically competent.

The above samples were analyzed by LC-MS/MS. Analysis was performed for the samples at each incubation solution. Results were determined by a comparison of peak ratios over the time course of the experiment (typically reported as “% Parent Remaining”).

Data were calculated with a LIMS (includes Galileo, Thermo Fisher Scientific Inc. and reporting tool, Crystal Reports, SAP), the spreadsheet computer program Microsoft Excel (Microsoft Corp.) or equivalent. The amount of unchanged parent compound was estimated (to determine approximate percent substrate remaining in each incubation) based on analyte/internal standard (IS) peak-area ratios using a LIMS, Analyst Instrument Control and Data Processing Software (AB SCIEX), or equivalent.

Results: Results as shown in FIG. 1 indicate that the EGE buprenorphine undergoes rapid metabolism in presence of microsomal enzymes for the duration of the assay. The microsomal enzymes are typically responsible for metabolism of drugs such as buprenorphine.

### Example 3

### Receptor Binding Activity

This example illustrates the binding of EGE buprenorphine hydrochloride to the μ-opioid receptor and x-opioid receptor.

### A. Human μ Opioid Receptor Binding Assay

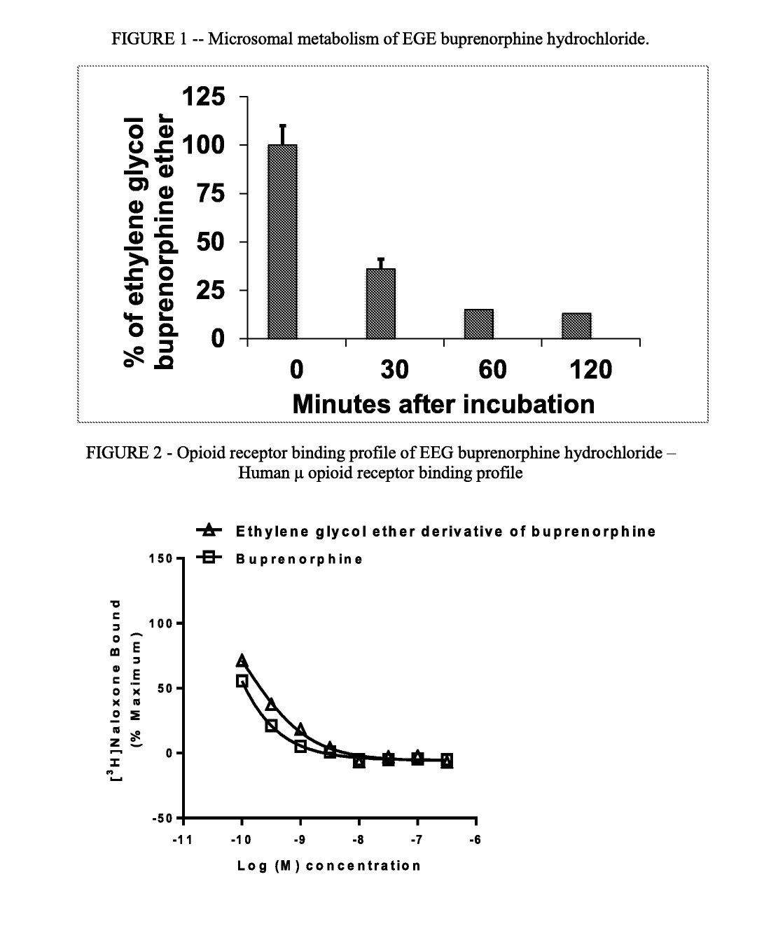

Membranes from Chinese Hamster Ovary cells expressing the human μ opioid receptor (Perkin Elmer #RBHOMM400UA) were homogenized in assay buffer (50 mM Tris, pH 7.5 with 5 mM MgCl2) using glass tissue grinder, Teflon pestle and Steadfast Stirrer (Fisher Scientific). The concentrates of the membranes were adjusted to 300 μg/mL in assay plate, a 96 well round bottom polypropylene plate. The compound to be tested was solubilized in DMSO (Pierce), 10 mM, then diluted in assay buffer to 3.6 nM. In a second 96 well round bottom polypropylene plate, known as the premix plate, 60 μL of 6× compound was combined with 60 μL of 3.6 nM 3H-Nalaxone. From the premix plate 50 μL was transferred to an assay plate containing the membranes, in duplicate. The assay plate was incubated for 2 h at room temperature. A GF/C 96 well filter plate (Perkin Elmer #6005174) was pretreated with 0.3% polyethylenimine for 30 min. The contents of the assay plate were filtered through the filter plate using a Packard Filtermate Harvester, and washed 3 times with 0.9% saline at 4° C. The filter plate was dried, underside sealed, and 30 μL Microscint 20 (Packard #6013621) was added to each well. A Topcount-NXT Microplate Scintillation Counter (Packard) was used to measure emitted energies in the range of 2.9 to 35 KeV. Results were compared to maximum binding, wells receiving no inhibitions. Nonspecific binding was determined in presence of 50 μM unlabeled naloxone. The biological activity of the EGE buprenorphine hydrochloride is shown in FIG. 2.

Results: The graphs in FIG. 2 show that EGE buprenorphine hydrochloride has significant affinity for the opioid μ receptor. The profile was similar to that of buprenorphine.

### B. Human κ Opioid Receptor Binding Assay

Membranes from cloned HEK-293 cells expressing the human kappa opioid receptor (Amersham Biosciences UK Ltd. 6110558 200U) were homogenized in assay buffer (50 mM Tris, pH 7.5 with 5 mM MgCl2) using glass tissue grinder, Teflon pestle and Steadfast Stirrer (Fisher Scientific). The concentrates of the membranes were adjusted to 300 μg/mL in assay plate, a 96 well round bottom polypropylene plate. The compound to be tested was solubilized in DMSO (Pierce), 10 mM, then diluted in assay buffer to 3.6 nM. In a second 96 well round bottom polypropylene plate, known as the premix plate, 60 μL of 6× compound was combined with 60 μL of 3.6 nM 3H-Diprenorphine (DPN). From the premix plate, 50 μL was transferred to an assay plate containing the membranes, in duplicate. The assay plate was incubated for 18 h at room temperature. A GF/C 96 well filter plate (Perkin Elmer #6005174) was pretreated with 0.3% polyethylenimine for 30 min. The contents of the assay plate were filtered through the filter plate using a Packard Filtermate Harvester, and washed 3 times with 0.9% saline at 4° C. The filter plate was dried, underside sealed, and 30 μL Microscint 20 (Packard #6013621) was added to each well. A Topcount-NXT Microplate Scintillation Counter (Packard) was used to measure emitted energies in the range of 2.9 to 35 KeV. Results were compared to maximum binding, wells receiving no inhibitions. Nonspecific binding was determined in presence of 50 μM unlabeled naloxone. The biological activity of the EGE buprenorphine hydrochloride is shown in FIG. 6.

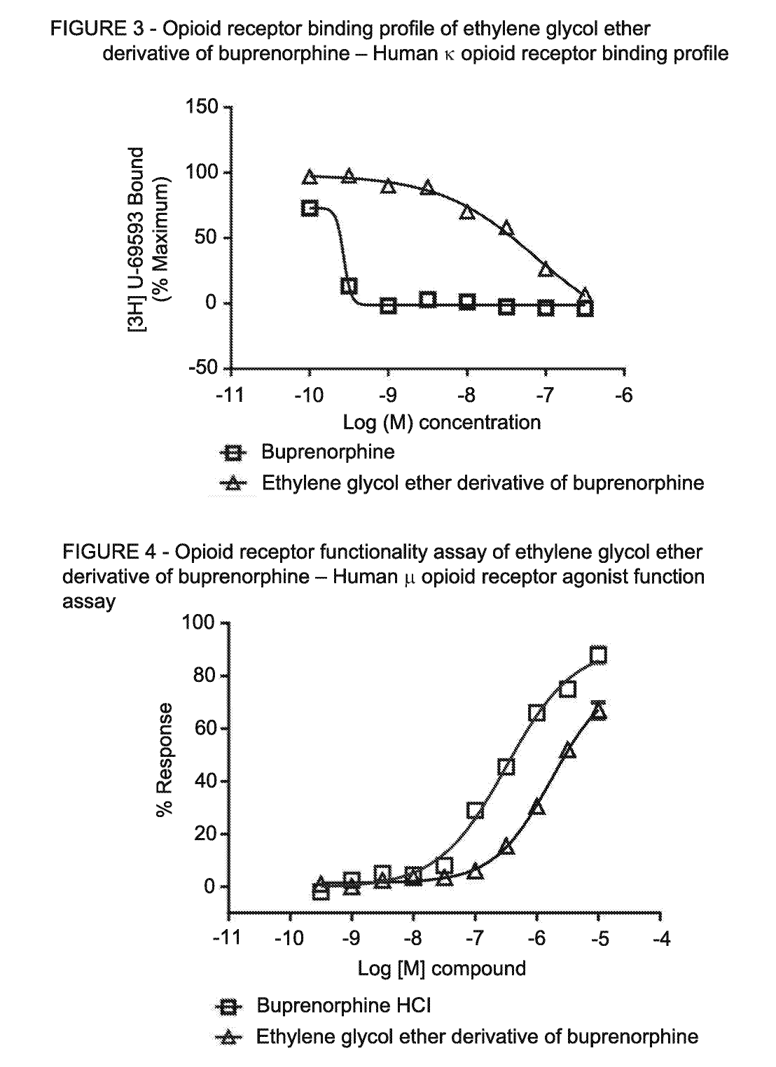

Results: FIG. 3 describes the opioid κ receptor agonist profile of the EGE buprenorphine hydrochloride and buprenorphine. Neither the EGE buprenorphine nor buprenorphine has lost its affinity for the κ receptor. Qualitatively, as with buprenorphine, the binding of the EGE to opioid κ receptor increases with concentration. It is estimated that at about 1 μg, the profile of the opioid κ receptor affinity of the EGE buprenorphine was similar to that of buprenorphine.

### Example 4

### Receptor Stimulation Activity

This example illustrates the ability of EGE buprenorphine hydrochloride to stimulate the μ-opioid receptor-mediated signaling.

### μ Opioid Receptor Agonist and Antagonist Functional Assays: [35S]GTPγS Binding Assay in Chinese Hamster Ovaries Expressing Human μ Receptors (CHO-hMOR) Cell Membranes

Briefly, CHO-hMOR cell membranes were purchased from Receptor Biology Inc. (Baltimore Md). About 10 mg/ml of membrane protein was suspended in 10 mM TRIS-HCl pH 7.2, 2 mM EDTA, 10% sucrose, and the suspension kept on ice. One mL of membranes was added to 15 mL cold binding assay buffer containing 50 mM HEPES, pH 7.6, 5 mM MgCl2, 100 mM NaCl, 1 mM DTT and 1 mM EDTA. The membrane suspension was homogenized with a polytron and centrifuged at 3000 rpm for 10 min. The supernatant was done centrifuged at 18,000 rpm for 20 min. The pellet was resuspended in 10 ml assay buffer with a polytron.

The membranes were pre-incubated with wheat germ agglutinin coated SPA beads (Amersham) at 25° C., for 45 min in the assay buffer. The SPA bead (5 mg/ml) coupled with membranes (10 μg/ml) were then incubated with 0.5 nM [35S]GTPγS in the assay buffer. The basal binding was that taking place in the absence of added test compound; this unmodulated binding was considered as 100%, with agonist stimulated binding rising to levels significantly above this value. A range of concentrations of receptor agonist SNC80 was used to stimulate [35S]GTPγS binding. Both basal and non-specific binding were tested in the absence of agonist; non-specific binding determination included 10 μM unlabeled GTPγS.

Buprenorphine and EGE buprenorphine hydrochloride were tested for function as an antagonist by evaluating their potential to inhibit agonist-stimulated GTPγS binding using D-Phe-Cys-Tyr-D-Trp-Orn-Thr-Pen-Thr-NH2 (CTOP) as the standard. Radioactivity was quantified on a Packard Top Count. The following parameters were calculated:

% Stimulation=[(test compound cpm−non-specific cpm)/(basal cpm−non-specific cpm)]*100% Inhibition=(% stimulation by 1 μM SNC80−% stimulation by 1 μM SNC80 in presence of test compound)*100/(% stimulation by 1 μM SNC80−100).

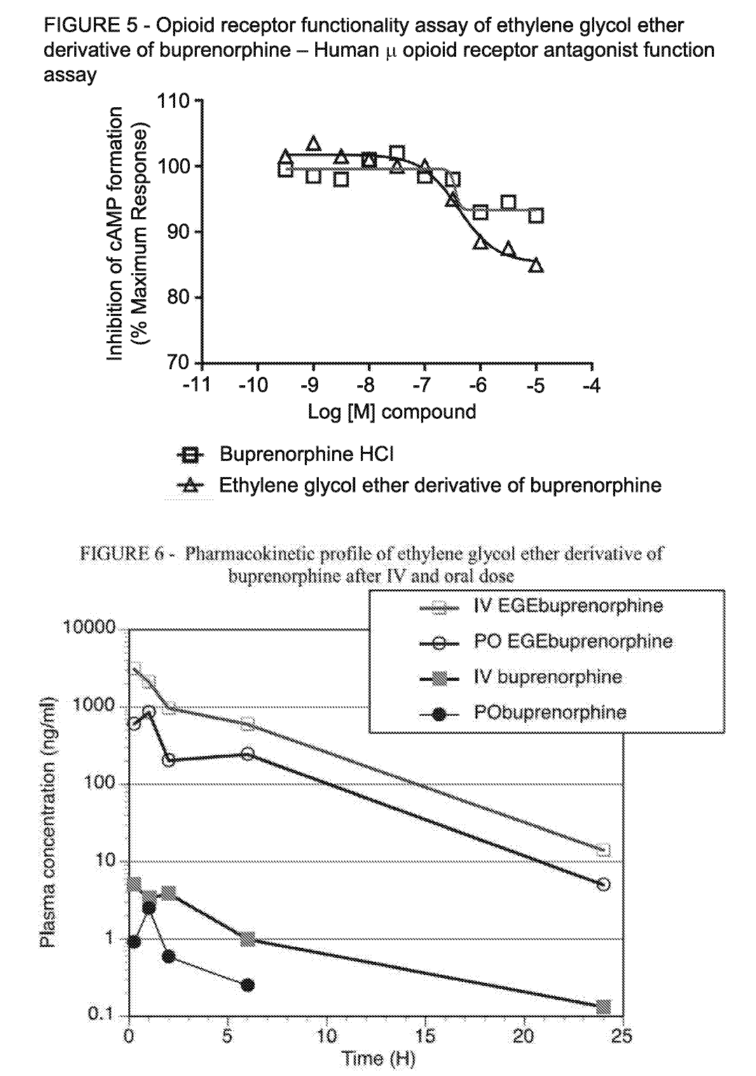

EC50 was calculated using GraphPad Prism. A graph for the compound tested is shown in FIGS. 4 and 5.

Results: Data shown in FIG. 4 indicates that the EGE bupreneorphine hydrochloride is a potent μ agonist. The results also indicate that the opioid μ receptor activity of the compound at 10-6M (˜1 μg) is similar to that of buprenorphine. Data in FIG. 5 shows that the EGE buorenorphine does not function as a μ-antagonist.

### Example 5

### In Vivo Pharmacokinetic Study

The animal pharmacokinetic studies were conducted at Johns Hopkins Medical Institute using CD-1 mice (weighing about 35 gm, n=3 per time point). PK analysis was conducted on buprenorphine and EGE buprenorphine hydrochloride that were each given at a dose of 10 mg/kg IV and by oral gavage. Blood collected at 0, 30 min and 1, 2, 6 and 24 hours post-dose. Blood samples for the drug were analyzed after harvesting the plasma and by LC/MS/MS as follows:

A standard curve was prepared in mouse plasma spiked with the test drug (10-25000 nM). Plasma samples (50 μL) were extracted in 300 μL acetonitrile containing losartan or buprenorphine-d4 as internal standard. Extracts were centrifuged at 16000×g at 4° C. for 5 minutes. Supernatants (250 μL) were transferred to a new tube and dried under N2 at 45° C. for 1 hour. Samples were reconstituted with 100 μL of 30% acetonitrile, vortexed and centrifuged. Supernatants (90 μL) were transferred to LC vials and 10 μL was injected on LC/MS. See FIG. 10.

Results: FIG. 6 depicts the plasma concentration profiles of the EGE buprenorphine after 10 mg oral and IV doses. The graph indicates that the absolute bioavailability of the dimer was about 40%, measured as a ratio of the area under the concentration curve after oral and IV dose.

### Example 6

### Stability of EGE Buprenorphine and Diethylene Glycol Ether Buprenorphine Conjugates

The purpose of the stability investigation was to determine the real time room temperature stability of the two agents over a period of time. The hydrochloride salts of the two conjugates were synthesized in May 2013 and stored in clear glass vials with polyseal cone caps. Stability was determined by comparing the purity the compound by HPLC immediately after synthesis and then in September 2015.

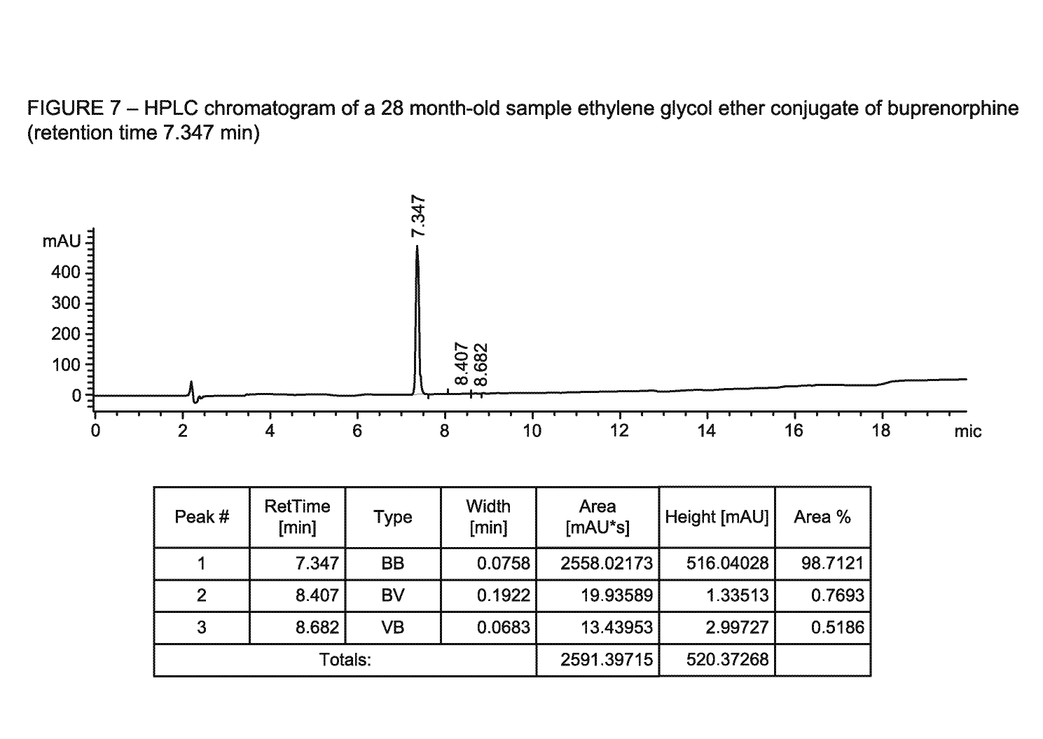

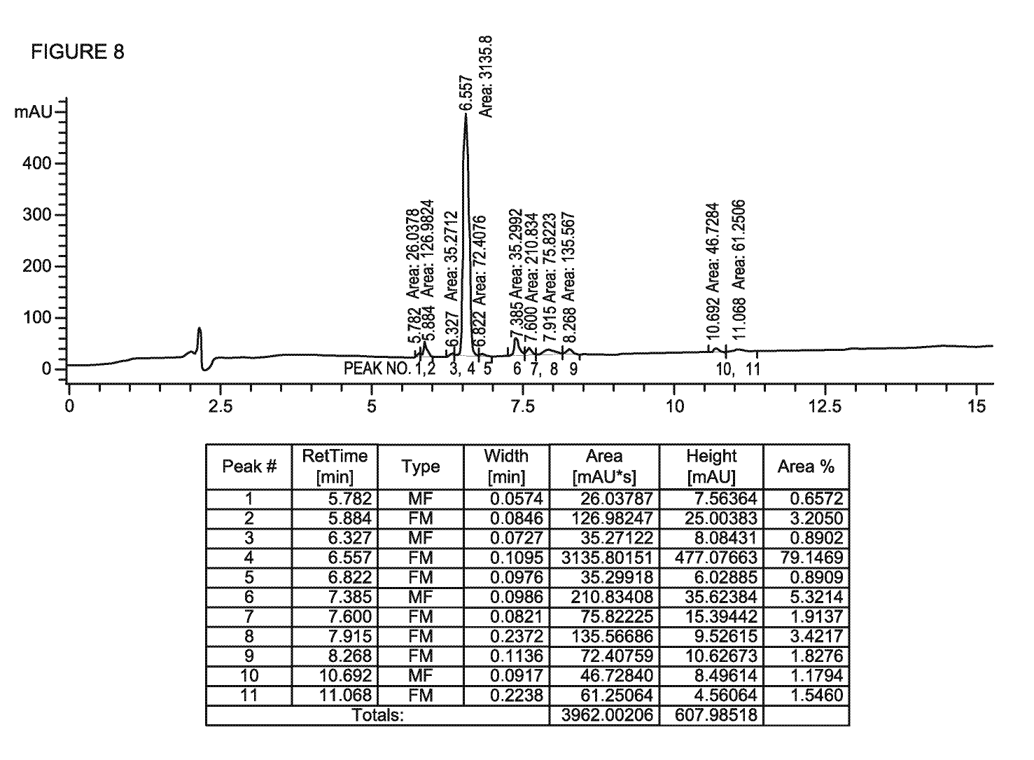

Method For HPLC analysis: 1 mg/ml of the two conjugates were dissolved in acetonitrile and 5 μL of it was injected on reverse phase C-18 column and the eluents were detected using a UV detector set at wavelength of 235 nm. The mobile system used for HPLC analysis was a gradient mixture of water containing 0.5% acetic acid and acetonitrile. Results in terms of peak purity of the two conjugates are shown in the Table below. Data shows that diethylene glycol ether conjugate (lot number MT-A-104-1) undergoes significant degradation over time after 28 months. In comparison to the diethylene glycol ether conjugate the EGE conjugate was unchanged over time. The HPLC chromatograms of the 2-year old samples are shown in FIGS. 7 and 8.

TABLE 3
Purity of EGE and diethylene glycol ether
conjugates of buprenorphine over 28 months.

| | HPLC Purity Over 28 Months | |
|---|---|---|
| Compound/Lot Number | May 2013 | September 2015 |
| Bup, Ethylene glycol ether., | 98.6% (AUC) | 98.7% (AUC) |
| (n = 1/MT-A-96-1) | | |
| Bup, Diethylene glycol ether | 95.6% (AUC) | 79.1% (AUC) |
| (n = 2/MT-A-104-1) | | |

### Example 7

### Analgesia—Formalin Paw Mouse Study

Male ICR mice weighing 23±3 g were divided into groups of 8 each. All test substances and vehicle control were administered intra-peritoneally in non-fasted mice prior to a sub-planter injection of formalin (0.02 ml 2% solution) administered to one hind paw. The hind paw licking time were recorded for about 35 minutes at 5 min intervals after formalin challenge as a measure of analgesic activity of the test compound compared to vehicle, acetaminophen and morphine.

TABLE 4
Study Design

| | | | Conc | Dose | Dose | Mice |
|---|---|---|---|---|---|---|
| Group | Test Article | Route | ng/ml | ml/kg | mg/kg | (male) |
| 1 | Vehicle | IP | n/a | 10 | n/a | 8 |
| 2 | Morphine | IP | 0.5 | 10 | 5 | 8 |
| 3 | Acetaminophen | IP | 20 | 10 | 200 | 8 |
| 5 | EGE | IP | 0.5 | 10 | 5 | 8 |
| | buprenorphine | | | | | |
| | hydrochloride | | | | | |

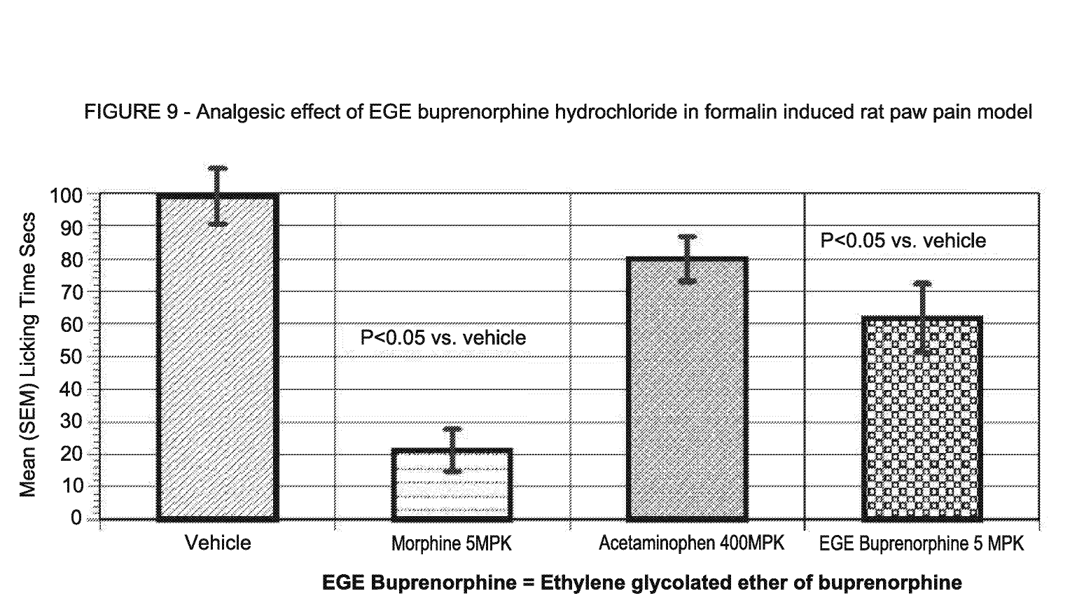

One-way ANOVA analysis followed by Dunnett's test was applied for comparison between vehicle control and test compound treated groups. The results are shown in FIG. 9. The compound of the invention exhibited a rapid analgesic effect.

### Example 8

### Oral Formulation

The following composition is exemplary of a representative oral tablet of the invention.

TABLE 5

| Ingredients | % w/w |
|---|---|
| EGE buprenorphine | 2 |
| hydrochloride | |
| Lactose | 83.6 |
| Colloidal Silicon dioxide | 0.67 |
| Microcrystalline cellulose | 10 |
| Croscarmellose sodium | 3.4 |
| Magnesium stearate | 0.33 |

### Example 9

### Abuse Deterrent Oral Formulation with an Inverse μ Agonist

The following composition is exemplary of a representative oral tablet of the invention in combination with an inverse μ opioid receptor agonist such as naloxone, naltrexone or the homo-dimers of these as described in our afore-mentioned U.S. patent application Ser. No. 14/697,155. Inverse μ agonists are also recognized in the literature as opioid receptor antagonists. A fixed combination with of these inverse agonists or antagonists will deter abuse of the invented compound because these compounds will prevent the invented compound from binding to and activating the μ opioid receptor. For optimum abuse deterrence the μ agonist, which is the compound of the invention, and the inverse μ agonist may be used in the ratio of about 1:1, 2:1, 3:1. Or 4:1.

TABLE 6

| Ingredients | % w/w |
|---|---|
| EGE buprenorphine | 2 |
| hydrochloride | |
| Naloxone | 1 |
| Lactose | 82.6 |
| Colloidal Silicon dioxide | 0.67 |
| Microcrystalline cellulose | 10 |
| Croscarmellose sodium | 3.4 |
| Magnesium stearate | 0.33 |

### Example 10

### Blender-Proof Immediate Release Oral Formulation

The following composition is exemplary of a representative tamper-proof oral tablet of the invention. Combination of the invented compound with one or more of the following polymers such as polysaccharides, sugars, sugar derived alcohols, starches, starch derivatives, cellulose derivatives, Carrageenan, pectin, sodium alginate, gellan gum, xanthan gum, poloxamer, Carbopol®, PolyOx®, povidone, hydroxypropyl methylcellulose (HPMC), hypermellose, and combinations thereof will prevent tampering because, when crushed, these polymers would gel in presence of moisture and thereby render the drug formulation unsuitable for snorting or injection. Ideally the polymers would be about 50% of the total formulation for long-acting, sustained-release medication and 10% for immediate release medication

TABLE 7

| Ingredients | % w/w |
|---|---|
| EGE buprenorphine | 2 |
| hydrochloride | |
| PolyOx ® | 10 |
| Lactose | 73.6 |
| Colloidal Silicon dioxide | 0.67 |
| Microcrystalline cellulose | 10 |
| Croscarmellose sodium | 3.4 |
| Magnesium stearate | 0.33 |

### Example 11

### Blender-Proof Sustained Release Oral Formulation

TABLE 8

| Ingredients | % w/w |
|---|---|
| EGE buprenorphine | 2 |
| hydrochloride | |
| PolyOx ® | 40 |
| Lactose | 43.6 |
| Colloidal Silicon dioxide | 0.67 |
| Microcrystalline cellulose | 10 |
| Croscarmellose sodium | 3.4 |
| Magnesium stearate | 0.33 |

It is understood that the examples and embodiments described herein are for illustrative purposes only and that various modifications or changes in light thereof will be suggested by persons skilled in the art and are to be included within the spirit and purview of this application and scope of the appended claims. To the extent there is conflict between the priority applications and the present application, any inconsistencies are to be resolved in favor of the present application. All publications and patents cited herein are hereby incorporated by reference in their entirety for all purposes.

What is claimed is:

1. A compound of the structure: 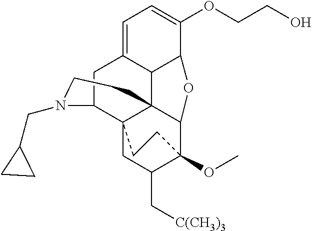
or a pharmaceutically acceptable solvate or salt thereof.

2. A composition comprising a compound according to claim 1 and a pharmaceutically acceptable excipient or carrier.

3. A composition according to claim 1 comprising a hydrochloride salt of the compound.

4. A composition according to claim 2 in the form of an oral tablet, capsule or film or extended release oral tablet, capsule or film.

5. A method for the treatment of chronic pain comprising orally administering to a subject a therapeutically effective amount of the compound of claim 1.

6. A method for the treatment of chronic pain comprising orally administering to a subject a therapeutically effective amount of a composition according to claim 2.

7. A method for the treatment of chronic pain comprising orally administering to a subject a therapeutically effective amount of a composition according to claim 4.

8. A method for treating chronic anxiety and depression comprising orally administering to a subject a therapeutically effective amount of a compound of claim 1 or a composition according to claim 4.
